(19) United States
(12) Patent Application Publication
(10) Pub. No.: US 2022/0000837 A1
(43) Pub. Date: Jan. 6, 2022

(54) Methods for Administration and Methods for Treating Cardiovascular Diseases with Resiniferatoxin

(71) Applicant: Board of Regents of the University of Nebraska, Lincoln, NE (US)

(72) Inventors: Irving H. Zucker, Omaha, NE (US); Hanjun Wang, Omaha, NE (US)

(73) Assignee: Board of Regents of the University of Nebraska, Lincoln, NE (US)

(21) Appl. No.: 17/375,796

(22) Filed: Jul. 14, 2021

Related U.S. Application Data
(63) Continuation of application No. 15/487,263, filed on Apr. 13, 2017, now abandoned.
(60) Provisional application No. 62/322,079, filed on Apr. 13, 2016.

(51) Int. Cl. A61K 31/357; A61K 9/00; A61K 45/06; A61K 31/4468

(52) U.S. Cl. CPC A61K 31/357; A61K 9/0085; A61K 9/0019; A61K 31/4468; A61K 45/06

(57) ABSTRACT

The present application provides methods for treating cardiovascular conditions. The methods can include administering a Transient Receptor Potential Vanilloid 1 (TRPV1) receptor agonist to an epidural space. The methods can be used to treat a variety of conditions such as hypertension, prehypertension, mild hypertension, severe hypertension, refractory hypertension, congestive heart failure and myocardial scarring.

### RELATED APPLICATIONS

This application is a Continuation of Application No. 15/487,263, filed April 13, 2017, which claims priority to U.S. Provisional Patent Application Ser. No. 62/322,079, filed Apr. 13, 2016, and entitled “Methods for Administration and Methods for Treating Cardiovascular Diseases with Resiniferatoxin,” the disclosure of which is hereby incorporated by reference in its entirety.

### GOVERNMENT SUPPORT

This invention was made with government support under Grant No. R01 HL126796 awarded by the National Institutes of Health. The government has certain rights in the invention.

### FIELD OF THE INVENTION

The present disclosure relates to ameliorative or preventative treatment of cardiovascular conditions, and provides methods for epidural administration of a formulation of a Transient Receptor Potential Vanilloid 1 (TRPV1), e.g., resiniferatoxin (RTX), to provide cardiac sympathetic afferent nerve ablation or denervation to treat or preventatively treat a patient with one or more cardiovascular conditions. The present disclosure provides for a method of administration of the formulation at one or more thoracic vertebral levels of the patient.

### BACKGROUND OF THE INVENTION

Cardiovascular conditions afflict a substantial number of subjects. Additionally, some cardiovascular conditions, including chronic heart failure, hypertension, and prehypertension, may themselves contribute to further cardiovascular damage and related degradation of a subject's health. Congestive heart failure (CHF) refers to a condition wherein the heart is unable to maintain sufficient blood flow throughout the cardiovascular system to meet its metabolic demands. Various treatments for CHF are known, including medications (e.g., diuretics, ACE inhibitors, beta blockers), medical devices (e.g., defibrillators), and life-style modification. Myocardial ischemia refers to a condition wherein the blood supply to the heart is insufficient, for example, due to blockage of the coronary arteries.

CHF is known to cause exaggerated activity of the sympathetic nervous system (Wang and Zucker (1996) Am J. Physiol. 271:R751-R756), and myocardial ischemia may also contribute to increased activity of the sympathetic nervous system (Zahner (2003) J. Physiol. 551.2:515-523) and activation of afferent (sensory) nerves (Wang et al. (2014) Hypertension 64(4):745-75). Increased and exaggerated sympatho-excitation and peripheral resistance may increase arterial pressure and heart rate. Over time, the increased arterial pressure and heart rate may contribute to further progression of chronic heart failure and damage to the cardiovascular system (Sing et al. (2000) Cardiovasc. Res. 45:713-719; Fowler et al. (1986) Circulation 74:1290-1302).

Hypertension is a condition wherein a subject exhibits chronic abnormally high arterial blood pressure. Hypertension may cause damage to the cardiovascular system, including thickening of arterial walls and hypertrophy of the left ventricle, and may contribute to CHF. A number of treatments for hypertension are known, including among them: lifestyle modification, diuretics, beta blockers, and ACE inhibitors. Some patients exhibit refractory hypertension (sometimes referred to as resistant hypertension or drug-resistant hypertension), which does not respond well to treatment by diuretics or other medication.

It is believed that increased activity of the cardiac sympathetic afferent reflex (CSAR) relates to the exaggerated sympatho-excitation observed in subjects with CHF (Wang (2000) Heart Fail. Rev. 5:57-71). Applicant has previously disclosed a method for reducing CSAR activity by chemically ablating or desensitizing CSAR-associated afferent endings or dorsal root ganglia (DRG) by treating the epicardium or DRG (U.S. Ser. No. 14/484,235).

Certain Transient Receptor Potential Vanilloid 1 (TRPV1) agonists, e.g. resiniferatoxin (RTX), exhibit an ability to desensitize DRG neurons. In particular, RTX exhibits strong and long-lasting neuron ablation, which has been studied, e.g., for pain management (Karai et al. (2004) J. Clin. Invest. 113:1344-13521; Szabo et al. (1999) Brain Res. 840:92-98). RTX may destroy neurons containing TRPV1 by inducing a calcium dependent toxic effect. A receptor within a neuron may be desensitized by application of a proper agonist, where the agonist triggers an acute response associated with pain followed by extended desensitization.

RTX is a phorbol-related diterpene, having a vanillyl substituent. The structure of RTX is depicted in FIG. 1. The vanillyl group permits RTX to function as a vanilloid receptor agonist, while the phorbol portion is believed to contribute to a substantial degree and duration of desensitizing effect (Szallasi et al. (1999) Brit. J. Pharmacol. 128:428-434). A number of similar compounds have been reported, each causing a varying degree of desensitization (Szallasi et al. (1999) Brit. J. Pharmacol. 128:428-434). RTX may be isolated from Euphorbia resinifera, and has also been synthesized (Wender et al. (1997) J. Am. Chem. Soc. 119:12976-12977).

Administration of medicinal formulations for cardiovascular treatment may be achieved in a number of ways. Epicardial and intrathecal administration, while useful for some subjects, present certain challenges. For example, injections to multiple locations and at a significant depth within the body may be complicated and time-consuming for a medical practitioner, and may cause discomfort and increased risk of injury to the subject. Intrathecal injections may be disfavored due to risk of harm from introduction of non-actives, e.g., preservatives or contaminates, into the spinal canal. The precautions necessary to reduce this risk may complicate preparation of formulations for intrathecal administration. In addition to these general disadvantages of epicardial and intrathecal administration, in the present context, i.e. nerve ablation or denervation within the spinal column, alternatives to intrathecal administration may be particularly desirable to avoid administering the formulation to the cerebrospinal fluid of the spinal canal. The cerebrospinal fluid permits high mobility, which may result in significant RTX migration through the spinal canal. This RTX migration may result in unnecessary denervation elsewhere in the spinal column, potentially affecting nerves unrelated to CSAR. Additionally, while epicardial administration exhibits a relatively long-lasting nerve afferent ablation, extending the duration of the effect is desirable for treatment of chronic cardiovascular conditions without the need for frequent repeated administration.

Therefore, there remains a need in the art for improved, targeted administration of a TRPV1 agonist, e.g. RTX, with reduced risks to patients to treat or prevent cardiovascular conditions.

### SUMMARY OF THE INVENTION

The present disclosure provides a method for epidural administration of a TRPV1 agonists, e.g., RTX, to provide cardiac sympathetic afferent ending denervation. The administration is to at least one of the first through fourth thoracic vertebrae. The disclosure provides a method for treating one or more cardiovascular conditions, including heart failure, hypertension and related indications selected from the group consisting of increased sympatho-excitation, cardiac hypertrophy, increased left ventricular end diastolic pressure (LVEDP), lung edema, and combinations thereof

Administration to the epidural space provides several advantages, including: less invasive treatment; greater ease of administration; and a reduction in potential adverse effects associated with intrathecal injection to the spinal canal or cardiac application (e.g., epicardial administration).

Epidural administration of RTX has also been found to achieve a more targeted nerve afferent ablation and a longer-lasting reduction in CSAR activity, as compared to epicardial administration. For example, in a rat model, CSAR activity remained reduced 6 months after epidural administration, as opposed to an increase in CSAR activity about 3-4 months after epicardial administration.

Further, administration to the epidural space may reduce unnecessary or undesired nerve ablation or denervation. Injection into the epidural space reduces the degree to which the formulation will migrate within the spinal column, as compared to administration to the spinal canal. The formulation will experience less mobility within the epidural space than in the spinal canal. The tissue within the epidural space will tend to retard the mobility of a formulation, as compared to greater mobility within the cerebrospinal fluid of the spinal canal.

Additionally, for certain embodiments of the present application, especially those in which administration is to a single epidural level, the number of injections is further reduced, as is the amount of unnecessary nerve ablation or denervation, as opposed to administration to each ganglion.

Additionally provided herein are methods of treating a cardiovascular condition in a patient, where the method provides for administering an opioid receptor agonist to the patient and administering a Transient Receptor Potential Vanilloid 1 (TRPV1) receptor agonist to an epidural space located at one or more of the first through fourth thoracic vertebral level of the patient. In embodiments, the opioid receptor agonist is an opioid and the opioid is fentanyl. The administration of the opioid receptor agonist may be, e.g., by intravenous administration or by intraperitoneal administration. The administration of the opioid receptor agonist may be made before the administration of the TRPV1 agonist. For example, the administration of the TRPV1 agonist may be made immediately subsequent to the administration of the opioid receptor agonist, or some period of time after the administration of the opioid receptor agonist, such as, for example, 1, 2, 3, 4, 5, 10, 15, 30, 45, 60 or 90 minutes after.

Opioid receptor agonists may include any compound that binds to and triggers the opioid receptor. Opioid receptor agonists include opioids. Opioids include both naturally-occurring and synthetic compounds, including, e.g., morphine, codeine, hydrocodone, oxycodone, fentanyl, and analogues thereof. One particular class of opioid receptor is the μ-opioid receptor. Specifically, μ-opioid receptor agonists include cebranopadol, eluxadoline, hydrocodone, hydromorphone, levorphanol, loperamide, methadone, nalbuphine, meperidine, tapentadol, codeine, DADLE, DAMGO, dihydromorphine, endomorphin-1, etonitazene, fentanyl, levomethadone, morphine, sufentanil, buprenorphine, butorphanol, (−)-pentazocine, alvimopan anhydrous, diprenorphine, levallorphan, methylnaltrexone, nalmefene, nalorphine, naloxone, naltrexone, naltriben, naltrindole, and quadazocine. As used herein, fentanyl may include analogues thereof, such as sufentanil, alfentanil, remifentanil, and lofentanil.

### BRIEF DESCRIPTION OF THE DRAWINGS

The invention will be more fully understood from the following detailed description taken in conjunction with the accompanying drawings, in which:

FIG. 1 shows the structure of resiniferatoxin (RTX).

FIG. 2 shows experimental data from a rat model showing the intensity of response of the TRPV1 receptor upon RTX injection at the indicated vertebral levels, i.e. , the first four thoracic vertebral levels, with accompanying images showing activity at the various vertebral levels.

FIG. 3A shows experimental data from a rat model showing isolectin B4 (IB4) and TRPV1 response for rat populations without RTX injection and with RTX injection.

FIG. 3B shows experimental data from a rat model showing the mean arterial pressure (MAP) and renal sympathetic nerve activity (RSNA) for a population without RTX treatment (vehicle) and a population with epidural RTX treatment, measured over a 26-week period.

FIG. 3C shows experimental data from a rat model showing MAP and RSNA for a population without RTX treatment (vehicle) and a population with epicardial RTX treatment, measured over a 26-week period.

FIG. 4 shows images of the dorsal horn of the spinal cord at T2 stained for both TRPV1 and Substance P (SP) comparing a subject that received RTX injection to a control subject.

FIG. 5 shows experimental data of cardiac function for each of four populations of Sprague-Dawley rats: sham rats with vehicle-only administration (column A), sham rats with epidural RTX administration (column B), rats with induced chronic heart failure (CHF) with vehicle-only administration (column C), and rats with induced chronic heart failure (CHF) with epidural RTX administration (column D), (n=9-16 for each group); the experimental data includes: body weight, heart weight, the ratio of heart weight to body weight (HW/BW), the ratio of wet lung weight to body weight (WLW/BW), the left ventricle end systolic pressure (LVESP), the left ventricle end diastolic pressure (LVEDP), maximum first derivative of left ventricular pressure (dp/dtₘₐₓ), the minimum first derivative of left ventricular pressure (dp/dtₘᵢₙ), and infarct size. Statistically significant values against the sham with vehicle population are indicated by an asterisk (*), and statistically significant values against the CHF with vehicle-only population are indicated by a dagger (†). Both significance measures were at the P<0.05 level.

FIG. 6 shows experimental data for cardiac function for each of four populations of Sprague-Dawley rats: sham rats with vehicle-only administration (column E), sham rats with epicardial RTX administration (column F), rats with induced chronic heart failure (CHF) with vehicle-only administration (column G), and rats with induced chronic heart failure (CHF) with epicardial RTX administration (column H), (n=20-25 for each group); the experimental data including: body weight, heart weight, the ratio of heart weight to body weight (HW/BW), the ratio of wet lung weight to body weight (WLW/BW), mean arterial pressure (MAP), the left ventricle end diastolic pressure (LVEDP), heart rate, the maximum first derivative of left ventricular pressure (dp/dtₘₐₓ), the minimum first derivative of left ventricular pressure (dp/dtₘᵢₙ), and infarct size. Statistically significant values against the sham with vehicle population are indicated by an asterisk (*), and statistically significant values against the CHF with vehicle-only population are indicated by a dagger (†). Both significance measures were at the P<0.05 level.

FIG. 7A shows experimental data from a rat model showing the long-term survival rate for rats with induced CHF without RTX treatment (n=20) and with epicardial RTX treatment (n=19) over a 28-week period.

FIG. 7B shows experimental data from a rat model showing the long-term survival rate for rats with induced CHF without RTX treatment (n=10) and with epidural RTX treatment at the first through fourth thoracic vertebral levels (n=9), over a 28-week period.

FIG. 8 shows experimental data from a rat model showing arterial blood pressure (ABP) and cardiac sympathetic nerve activity (CSNA) for sham rats without treatment, rats with induced CHF without treatment, rats with induced CHF with epicardial RTX treatment, and rats with induced CHF with epidural RTX.

FIG. 9 shows experimental data from a rat model showing basal cardiac sympathetic tone for cardiac sympathetic nerve activity (CSNA) and renal sympathetic nerve activity (RSNA) for sham and vehicle-only, sham and RTX, CHF and vehicle-only, and CHF with RTX populations. In each case, administration of vehicle or RTX and vehicle was epidural. Statistically significant values against the sham with vehicle population are indicated by an asterisk (*), and statistically significant values against the CHF with vehicle-only population are indicated by a number sign (#). Both significance measures were at the P<0.05 level.

FIG. 10 shows experimental data from a rat model showing end-systolic pressure volume relationship (ESPVR) for sham and vehicle-only, sham and RTX, CHF and vehicle-only, and CHF with RTX administration populations. In each case, administration of vehicle or RTX and vehicle was epidural. Statistically significant values (at the P<0.05 level) against the sham with vehicle population are indicated by an asterisk (*).

FIG. 11 shows experimental data from a rat model showing the end diastolic pressure volume relationship (EDPVR) for sham and vehicle-only, sham and RTX, CHF and vehicle-only, and CHF with RTX populations. In each case, administration of vehicle or RTX and vehicle was epidural. Statistically significant values against the sham with vehicle population are indicated by an asterisk (*), and statistically significant values against the CHF with vehicle-only population are indicated by a number sign (#). Both significance measures were at the P<0.05 level.

FIG. 12 shows experimental data from a rat model showing the mean arterial pressure (MAP) over 24 hours for sham and vehicle-only, sham and RTX, CHF and vehicle-only, and CHF with RTX administration populations (n=5-8). The study was conducted 10-12 weeks after the myocardial infarction.

FIG. 13A shows mean arterial pressure (MAP) from experimental data from a rat model featuring early hypertensive (i.e., prehypertensive or mildly hypertensive) subjects, both with RTX treatment and a control population with vehicle treatment only. Open circles indicate the control population, while filled circles indicate the RTX-treated population. Epidural administration of RTX was by injection on Day 0, as indicated by the arrow on the graph. The asterisk (*) and bar indicate data significantly different as between the two populations.

FIG. 13B shows systolic arterial pressure from experimental data from a rat model featuring early hypertensive (i.e., prehypertensive or mildly hypertensive) subjects, both with RTX treatment and a control population with vehicle treatment only. Open circles indicate the control population, while filled circles indicate the RTX-treated population. Epidural administration of RTX was by injection on Day 0, as indicated by the arrow on the graph. The asterisk (*) and bar indicate data significantly different as between the two populations.

FIG. 13C shows diastolic arterial pressure from experimental data from a rat model featuring early hypertensive (i.e.,prehypertensive or mildly hypertensive) subjects, both with RTX treatment and a control population with vehicle treatment only. Open circles indicate the control population, while filled circles indicate the RTX-treated population. Epidural administration of RTX was by injection on Day 0, as indicated by the arrow on the graph. The asterisk (*) and bar indicate data significantly different as between the two populations.

FIG. 14A shows mean arterial pressure (MAP) from experimental data from a spontaneously hypertensive rat (SHR) model with established hypertension, including a population with RTX treatment and a control population with vehicle treatment only. Open circles indicate the control population, while filled circles indicate the RTX-treated population. Epidural administration of RTX was by injection on Day 0, as indicated by the arrow on the graph. In each case, the asterisk (*) and bar indicate data significantly different as between the two populations.

FIG. 14B shows systolic arterial pressure from experimental data from a spontaneously hypertensive rat (SHR) model with established hypertension, including a population with RTX treatment and a control population with vehicle treatment only. Open circles indicate the control population, while filled circles indicate the RTX-treated population. Epidural administration of RTX was by injection on Day 0, as indicated by the arrow on the graph. In each case, the asterisk (*) and bar indicate data significantly different as between the two populations.

FIG. 14C shows diastolic arterial pressure from experimental data from a spontaneously hypertensive rat (SHR) model with established hypertension, including a population with RTX treatment and a control population with vehicle treatment only. Open circles indicate the control population, while filled circles indicate the RTX-treated population. Epidural administration of RTX was by injection on Day 0, as indicated by the arrow on the graph. In each case, the asterisk (*) and bar indicate data significantly different as between the two populations.

FIG. 15A shows mean arterial pressure (MAP) in mmHg, from experimental data from an established hypertensive rat model treated with RTX via lumbar administration in the L2-L5 region showing a period of seven days before administration and 60 days after administration, with RTX administration made on Day 0. Blood pressure measurements are 8 hours average per day.

FIG. 15B shows heart rate (beats per minute) from experimental data from an established hypertensive rat model treated with RTX via lumbar administration in the L2-L5 region showing a period of seven days before administration and 60 days after administration, with RTX administration made on Day 0. Blood pressure measurements are 8 hours average per day.

FIG. 16 shows the ambulatory blood pressure (ABP), MAP, and heart rate of a hypertensive rat over time, showing the periods before, during, and after administration of RTX. The time of administration of RTX at each of the T1-T4 vertebral levels is indicated by asterisks.

FIG. 17 shows the ambulatory blood pressure (ABP), MAP, and heart rate of a hypertensive rat over time for a subject that received pre-treatment with 7 μg/kg intravenous fentanyl, showing the periods before, during, and after administration of RTX. The time of administration of RTX at each of the T1-T4 vertebral levels is indicated by asterisks.

FIG. 18 shows comparative data for three hypertensive rat populations, where a first group (n=9) received no pre-treatment (RTX-only), a second group (n=7) received pre-treatment with 20 μg/kg intraperitoneal fentanyl (RTX +IP Fen (20 μg/kg)), and a third group (n=5) received pre-treatment with 3.5 μg/kg intravenous fentanyl (IV Fen (3.5 μg/kg)). The data shows the change in MAP and heart rate over baseline measurements.

FIG. 19 shows comparative data for three hypertensive rat populations, where a first group (n=9) received no pre-treatment (RTX-only), a second group (n=7) received pre-treatment with 20 μg/kg intraperitoneal fentanyl (RTX+IP Fen (20 μg/kg)), and a third group (n=5) received pre-treatment with 3.5 μg/kg intravenous fentanyl (IV Fen (3.5 μg/kg)). The data shows MAP for each population before treatment (baseline), after pre-treatment for the pre-treated populations (designated “Fen”), and following RTX treatment.

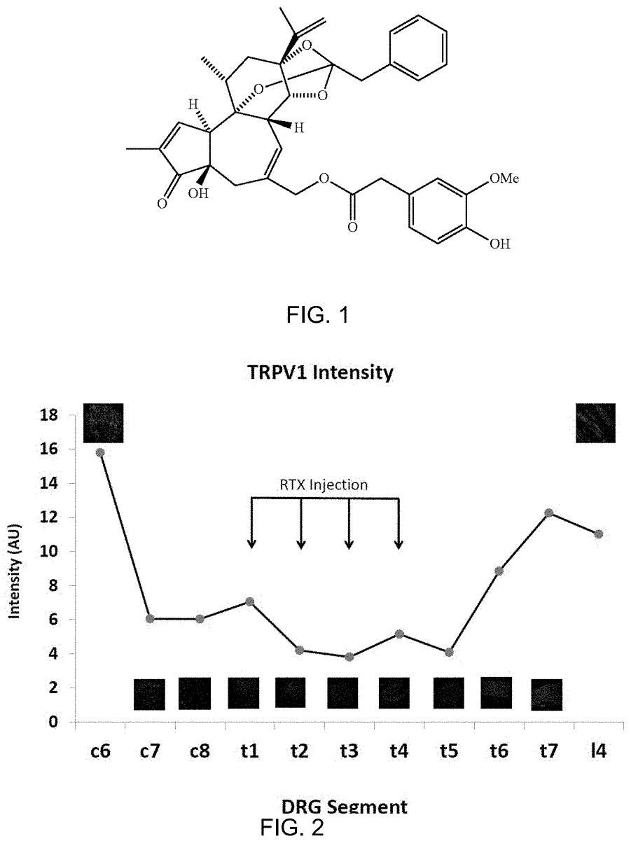

### DESCRIPTION OF EXEMPLARY EMBODIMENTS

The present disclosure relates to a method of treating cardiovascular condition(s) in a subject, the method including administering a Transient Receptor Potential Vanilloid 1 (TRPV1) agonist to an epidural space in at least one of a first through fourth thoracic vertebral level of the patient. In certain embodiments, the cardiovascular condition may be congestive heart failure. Alternatively, the cardiovascular condition may be scarring of the patient's myocardium. Alternatively, the cardiovascular condition may be selected from a group consisting of hypertension, prehypertension and mild hypertension. The cardiovascular condition may include severe hypertension or drug-resistant or refractory hypertension.

Additionally, application of the TRPV1 agonist in accordance with the present disclosure may be made to a single vertebral level. In certain embodiments, the TRPV1 agonist may be administered to the epidural space proximate a first thoracic vertebra; the TRPV1 agonist may be administered to the epidural space proximate a second thoracic vertebra; the TRPV1 agonist may be administered to the epidural space proximate a third thoracic vertebra; or the TRPV1 agonist may be administered to the epidural space proximate a fourth thoracic vertebra.

The present disclosure additionally relates to a method of treating a cardiovascular condition in a subject, the method including administering resiniferatoxin (RTX) to an epidural space in at least one of the first through fourth thoracic vertebral levels of the patient. In certain embodiments, the cardiovascular condition may be congestive heart failure. Alternatively, the cardiovascular condition may be scarring of the patient's myocardium, Alternatively, the cardiovascular condition may be selected from a group consisting of hypertension, prehypertension, or mild hypertension. Alternatively, the cardiovascular condition may be selected from a group consisting of severe hypertension or refractory hypertension. In certain embodiments, the RTX may be administered to the epidural space proximate to the first thoracic vertebra; the RTX may be administered to the epidural space proximate a second thoracic vertebra; the RTX may be administered to the epidural space proximate a third thoracic vertebra; or the RTX may be administered to the epidural space proximate a fourth thoracic vertebrae.

The present disclosure relates to a method of preventative treatment of a subject, the subject having pre-hypertension or mild hypertension, the method including administering a TRPV1 agonist to an epidural space near a thoracic vertebra of the subject.

The present disclosure relates, in some embodiments, to a method of preventative treatment of a subject, the subject having pre-hypertension or mild hypertension, the method including administering RTX to an epidural space near a thoracic vertebra of the subject.

The present disclosure additionally relates, in some embodiments, to a method of treating a cardiovascular condition in a patient, the method including administering an amount of RTX to an epidural space at one or more of the first through fourth thoracic vertebral levels of the patient, the amount being more than about 0.06 μg and less than about 30 μg.

The present disclosure additionally relates, in some embodiments, to a method of treating a cardiovascular condition in a patient, said method including administering a solution to an epidural space at one or more of the first through fourth thoracic vertebral levels of the patient, the solution including 0.6 to 10 μg of RTX per milliliter (mL) of solution. In certain embodiments, the solution may be administered at a volume of more than about 100 mL and less than about 3 mL at each of said one or more vertebral levels.

With respect to a human subject, the term “hypertension” means a condition wherein a patient exhibits either or both of: (i) systolic blood pressure at or above 140 mm Hg, and (ii) diastolic blood pressure at or above 90 mm Hg. The terms “prehypertension” or “mild hypertension” mean a condition wherein a patient exhibits either or both of: (i) systolic blood pressure at or above 120 mm Hg but below 140 mm Hg, and (ii) diastolic blood pressure at or above 80 mm Hg, but below 90 mm Hg. The term “severe hypertension” means a condition wherein a patient exhibits either or both of: (i) systolic blood pressure at or above 180 mm Hg, and (ii) diastolic blood pressure at or above 110 mm Hg. With respect to a nonhuman subject, the terms “hypertension,” “prehypertension,” “mild hypertension,” and “severe hypertension” mean sustained blood pressures in the nonhuman subject, equivalent to the foregoing in a human subject. The term “resistant hypertension” refers to a condition wherein a subject's blood pressure remains above a goal blood pressure for that patient during concurrent treatment with at least three anti-hypertensive agents of different classes, wherein each anti-hypertensive agent is prescribed at optimal dosage amount and, preferably, at least one of the three agents is a diuretic.

The term “epidural space” means any part of the space between a vertebra and the dura mater of the spinal column. The term “vertebral level” means a vertebra and the portions of the spinal column proximate to that vertebra. A vertebral level includes spaces interior to the vertebrae, including the epidural space, dura mater, and spinal cord.

Vertebral levels may be designated numerically, wherein the first thoracic vertebral level is the vertebral level proximate the cervical vertebra and is the thoracic vertebra closest to the skull. According to that designation, the vertebral level numbering then proceeds down the spine toward the lumbar vertebrae. The thoracic vertebrae are designated T1-T12.

The nerves associated with the sympatho-excitation corresponding to increased cardiac sympathetic nerve activity are predominately found within the first four thoracic vertebral levels (Evans et al. (1953) N. Engl. J. Med. 249:791-796). Thus, administration to one or more of the first four thoracic vertebral levels may be beneficial because the nerves associated with cardiac sympathetic afferent nerve activity are predominately found within the first four thoracic vertebral levels. By treating these thoracic vertebral levels, denervation of these sympathetic afferent nerves will be substantially achieved with minimal or only partial denervation or ablation of nerves at other vertebral levels. FIG. 2 shows the response of the TRPV1 receptor following RTX injection at the indicated vertebral levels (T1-T4). FIG. 2 demonstrates that, when administration is within the first four thoracic vertebral levels, the reduced vanilloid receptor intensity is generally localized to the four treated vertebral levels and the one or two vertebral levels proximate the treated vertebral levels. Additionally, the small images inset on the figure show greater intensity of activity visible at the C6, C7, T6, T7, and L4 vertebral levels, as compared to the C8 through T5 vertebral levels.

Administration to the epidural space may be by one, two, or more injections at each vertebral level. Where two injections are given, the two injections may consist of one injection on each side of a vertebra (i.e., a bilateral injection). In an embodiment with a human subject, the administration may occur in phases: in a first phase, the patient may receive unilateral injection at one or two vertebral levels (either right or left side) followed by a short period of recovery. Thereafter, if sufficient effectiveness is not observed, a subsequent phase of administration may be provided with injections at the other side of the treated vertebral levels, at other vertebral levels, or both. The administration alternatively may be through a medical implant/device. In a rat model, administration has been made through a catheter inserted into the subarachnoid space at the T13-L1 thoracic vertebral region, advanced to the T1 level, and then pulled back, with serial injections made at the desired vertebral levels.

In an embodiment, a human subject may receive epidural injection of RTX (0.6-10 μg/mL, 100 μL-1.5 mL on each side at each vertebral level), using fluoroscopy for guidance, by insertion of a needle into the skin and directed toward the epidural space for injection into the epidural space. In an embodiment, the patient may initially be treated by injection at just one side at one or more vertebral levels. Subsequently, if a large effect is required, the patient may be treated by injection at the other side of that level or at additional levels. Treatment at one side of a vertebral level may predominantly treat a single ganglion. Beginning treatment with a limited number of ganglia and then increasing treatment, if required, may control side effects associated with nerve ablation.

Furthermore, the RTX may be delivered concurrently, after, or just before administration of other drugs. For example, administration of certain opioids or other agents close to the time of RTX delivery may reduce or blunt short-term adverse changes in BP and/or HR. Accordingly, opioids such as fentanyl or other drugs may be given via known routes, e.g., IV, oral, buccal, peritoneal, rectal, or other routes.

The present disclosure may be further understood by reference to the following Examples. It should be understood that these Examples, while indicating various embodiments of the disclosure, are given by way of illustration only.

### EXAMPLES

### Treatment of Chronic Heart Failure Subjects.

The following Examples 1 through 9 discuss data from rats with induced CHF. The Examples show many improved indicators associated with RTX treatment in CHF subjects. The rat models provide for comparison of epidural administration of RTX against epicardial administration of RTX and against various controls. For the epicardial data, RTX was administered directly to the epicardium by swab during open surgery contemporaneously with induced myocardial infarction (coronary artery ligation).

For epidural application of RTX, a small midline incision was made in the region of the T13-L1 thoracic vertebrae. Following dissection of the superficial muscles, two small holes (approximately 2 mm by 2 mm) were made in the left and right sides of the T13 vertebra. A polyethylene catheter (PE-10) was inserted into the subarachnoid space via the left hole and gently advanced about 5.5-6 cm to the left T1 level at which the first injection (6 μg/mL, 10 μL) was made at a very slow speed to minimize the diffusion of RTX. Then the catheter was pulled back about 0.5 cm to left T2, T3, and T4, respectively, to perform serial injections (10 μL each) in each segment. Then the catheter was withdrawn and the same injection was repeated on the right side. A silicone gel was used to seal the hole in the T13 vertebra. The skin overlying the muscle was closed with a 3-0 polypropylene suture. Simple interrupted sutures were used to close the skin.

### Example 1

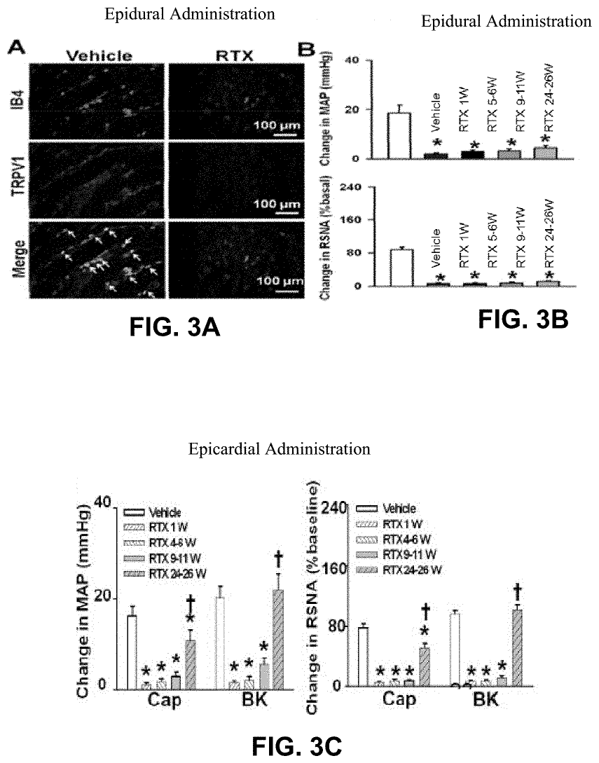

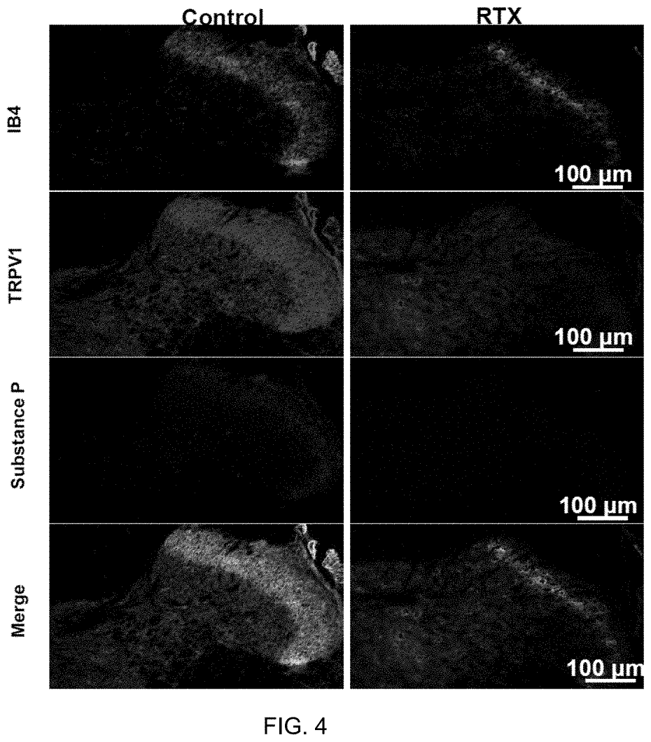

FIGS. 3A-4B show experimental results for a rat study in accordance with an embodiment of the present application. FIG. 3A shows experimental data from a rat model showing isolectin B4 (IB4) and TRPV1 response for rat populations without epidural RTX injection and with epidural RTX injection. FIG. 3B shows experimental data from a rat model showing the mean arterial pressure (MAP) and renal sympathetic nerve activity (RSNA) for a population without RTX treatment (vehicle) and a population with epidural RTX treatment, measured over a 26 week period. FIG. 3C shows experimental data from a rat model showing MAP and RSNA for a population without RTX treatment (vehicle) and a population with epicardial RTX treatment, measured over a 26 week period.

FIG. 3A shows a reduction in isolectin B4 (IB4) and TRPV1 expression in DRG neurons of various sizes following RTX treatment as compared to the vehicle-only treatment, which shows visually-apparent increased expression. FIG. 3B shows the response to activation of the CSAR for populations with and without RTX treatment. FIG. 3B shows that the effects of RTX upon mean arterial pressure (MAP) and renal sympathetic nerve activity (RSNA) are of extended duration. Following epidural RTX administration, both MAP and RNSA exhibit a substantial decrease compared to control rats without RTX treatment (vehicle). The substantial decrease is observed at: weeks 1, 5-6, 9-11, and 24-26.

FIG. 3C shows experimental data for a rat study using epicardial administration of RTX. FIG. 3C shows that cardiac sympathetic afferent ablation following epicardial RTX administration had a duration of only about 3-4 months. FIG. 3B shows that cardiac sympathetic afferent ablation following epidural administration of RTX had a duration of at least 6 months.

### Example 2

FIG. 4 shows experimental results for a rat study in accordance with an embodiment of the present application. FIG. 4 shows images of the dorsal horn of the spinal cord at T2 stained for both TRPV1 and Substance P (SP) comparing a subject that received RTX injection to a control subject. Epidural application of RTX at the T1-T4 DRG levels ablated almost all SP-containing C fiber afferents (peptidergic) and a large portion of isolectin B4 (IB4)-positive C fiber afferents (non-peptidergic) that project to the dorsal horn of the thoracic spinal cord. FIG. 4 shows reduced expression of TRPV1 protein and destruction of IB4 containing cell bodies, suggesting that small diameter neurons were ablated. Neurons in the dorsal horn of the spinal cord that express SP were ablated by RTX. IB4 is an indicator of small diameter afferent nerves and SP is an indicator of neuroinflammation. In each case, the reduced expression is visibly apparent from the reduced signal in the images from the RTX-treated subject, as compared to the control subject.

### Example 3

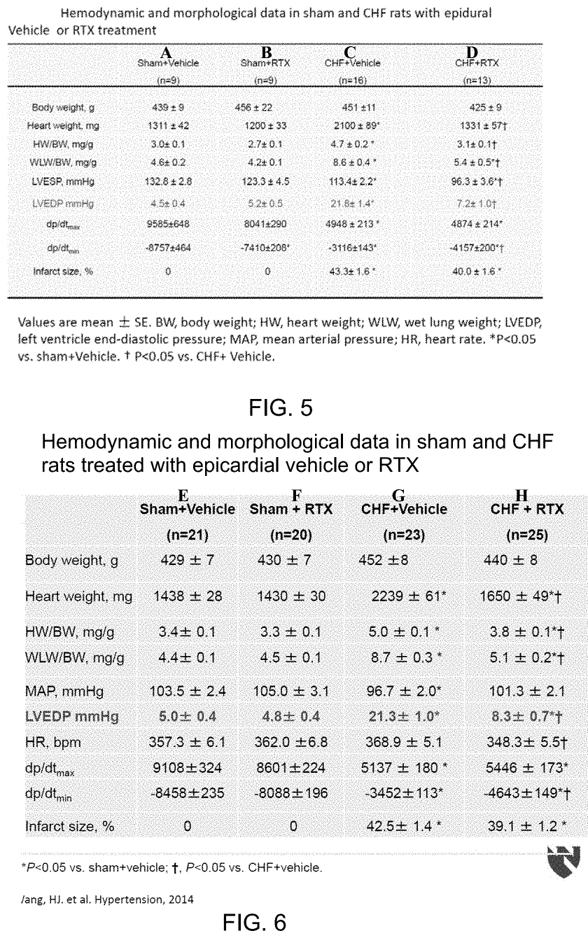

FIG. 5 shows experimental results for a rat study in accordance with an embodiment of the present application. FIG. 5 shows experimental data of cardiac function for each of four populations of Sprague-Dawley rats: sham rats with vehicle-only administration (column A), sham rats with epidural RTX administration (column B), rats with induced chronic heart failure (CHF) with vehicle-only administration (column C), and rats with induced chronic heart failure (CHF) with epidural RTX administration (column D) (n=9-16 for each group). The experimental data included: body weight, heart weight, the ratio of heart weight to body weight (HW/BW), the ratio of wet lung weight to body weight (WLW/BW), the left ventricle end systolic pressure (LVESP), the left ventricle end diastolic pressure (LVEDP), maximum first derivative of left ventricular pressure (dp/dtₘₐₓ), the minimum first derivative of left ventricular pressure (dp/dtₘᵢₙ), and infarct size. Statistically significant values against the sham with vehicle population are indicated by an asterisk (*), and statistically significant values against the CHF with vehicle-only population are indicated by a dagger (†). Both significance measures were at the P<0.05 level. The column titled “CHF+vehicle” shows various indicators for control rats with induced chronic heart failure. The column titled “CHF+RTX” shows various indicators for rats with induced chronic heart failure that received epidural RTX injections. The columns titled “Sham+vehicle” and “Sham+RTX” provide a comparison for sham rats without induced chronic heart failure, and without and with, respectively, epidural RTX treatment.

Comparison of columns C and A shows the following statistically significant effects of chronic heart failure on the tested rat population: a substantial increase in heart weight, heart weight as a percentage of body weight (consistent with cardiac hypertrophy), wet lung weight as a percentage of body weight (consistent with pulmonary congestion), and left ventricle end diastolic pressure; and a substantial decrease (in absolute terms) of the maximum first derivative of left ventricular pressure (dp/dtₘₐₓ), and the minimum first derivative of left ventricular pressure (dp/dtₘᵢₙ), which indicate reduced myocardial contractility. Each of these results is consistent with weakening of the heart expected in chronic heart failure. Column D shows that the population that received epidural administration of RTX exhibited statistically significantly better cardiovascular function with respect to: heart weight, heart weight as a percentage of body weight, wet lung weight as a percentage of body weight, left ventricle end diastolic pressure, and minimum first derivative of left ventricular pressure (dp/dtₘᵢₙ). In particular, left ventricle end diastolic pressure, which exhibited a 380% increase in the population with chronic heart failure over sham, showed only a 60% increase after RTX treatment. The similarity in infarct size observed in Columns C and D—the difference not being statistically significant—suggests that the improved results cannot be explained by the size of the infarct in the subject.

FIG. 6 shows treatment with RTX by epicardial administration for comparison. FIG. 6 shows experimental data for cardiac function for each of four populations of Sprague-Dawley rats: sham rats with vehicle-only administration (column E), sham rats with epicardial RTX administration (column F), rats with induced chronic heart failure (CHF) with vehicle-only administration (column G), and rats with induced chronic heart failure (CHF) with epicardial RTX administration (column H) (n=20-25 for each group). The experimental data included: body weight, heart weight, the ratio of heart weight to body weight (HW/BW), the ratio of wet lung weight to body weight (WLW/BW), mean arterial pressure (MAP), the left ventricle end diastolic pressure (LVEDP), heart rate, the maximum first derivative of left ventricular pressure (dp/dtₘₐₓ), the minimum first derivative of left ventricular pressure (dp/dtₘᵢₙ), and infarct size. Statistically significant values against the sham with vehicle population are indicated by an asterisk (*), and statistically significant values against the CHF with vehicle-only population are indicated by a dagger (†). Both significance measures were at the P<0.05 level.

Comparison of FIG. 6 column H with FIG. 5 column D reveals that the results achieved by epidural administration were comparable to the results achieved by epicardial administration, or are in some cases better, e.g., left ventricular end diastolic pressure. As discussed above, epidural treatment provides a number of advantages over epicardial treatment related to the ease of administration and potential side effects of the treatment, while also providing lasting physiologic effects compared to the transient effects associated with epicardial treatment. Thus, for some patients, an epidural treatment having at least comparable efficacy to an epicardial treatment is preferable.

### Example 4

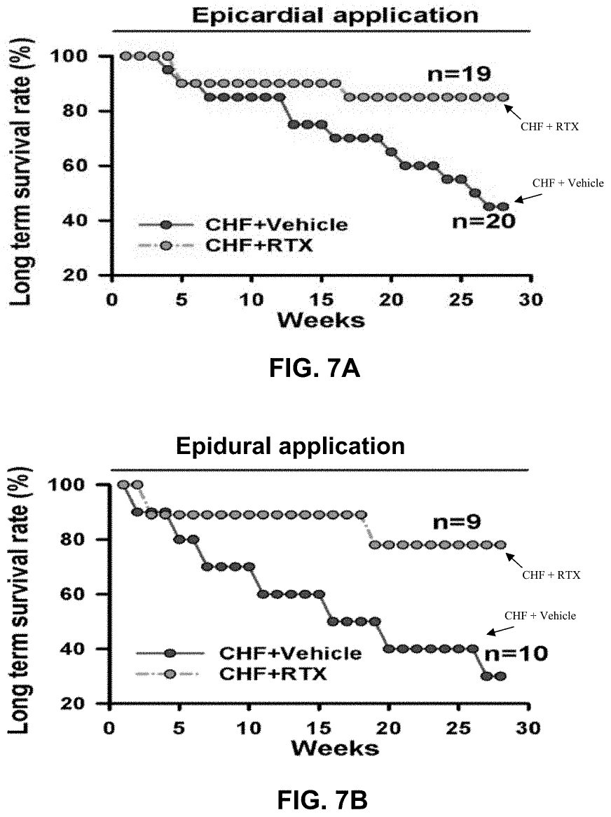

FIG. 7 shows experimental results for a rat study in accordance with an embodiment of the present application. FIG. 7A shows experimental data from a rat model showing the long-term survival rate for rats with induced CHF without RTX treatment (n=20) and with epicardial RTX treatment (n=19) over a 28-week period. FIG. 7B shows experimental data from a rat model showing the long-term survival rate for rats with induced CHF without RTX treatment (n=10) and with epidural RTX treatment at the first through fourth thoracic vertebral levels (n=9), over a 28-week period. FIG. 7 depicts the survival rate of rats with induced chronic heart failure with and without RTX treatment. In FIG. 7A, the treatment was epicardial. In FIG. 7B the treatment was epidural. As shown in FIG. 7B, the long-term survival rate of rats treated with epidural RTX was significantly higher than the survival rate for rats not treated by RTX. In particular, without RTX treatment, seven of ten rats died during the 28-week period. But only two of nine rats with RTX treatment died during the same period. Additionally, epidural treatment showed a roughly comparable improvement as reported for epicardial treatment.

### Example 5

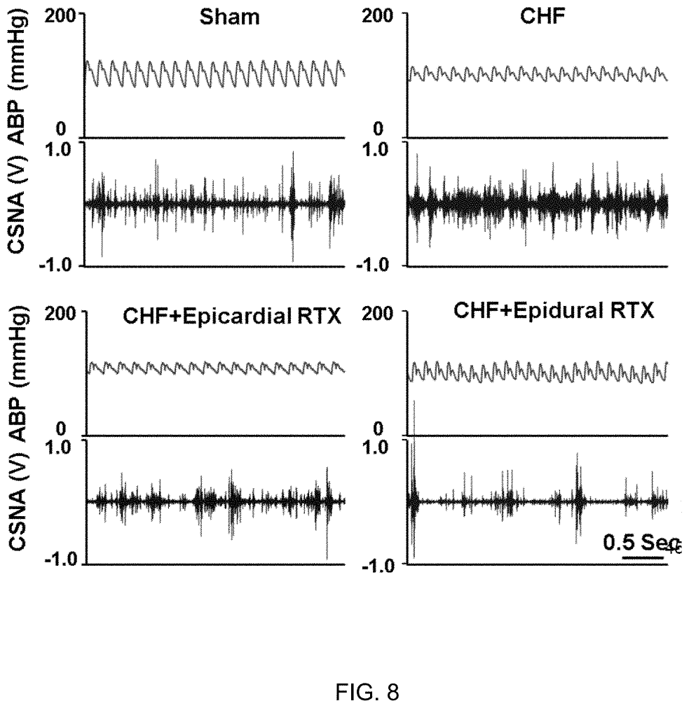

FIG. 8 shows experimental results for a rat study in accordance with an embodiment of the present application. FIG. 8 shows experimental data from a rat model showing arterial blood pressure (ABP) and cardiac sympathetic nerve activity (CSNA) for sham rats without treatment, rats with induced CHF without treatment, rats with induced CHF with epicardial RTX treatment, and rats with induced CHF with epidural RTX treatment. FIG. 8 shows increased CSNA with CHF, and shows that while both epicardial and epidural RTX treatment exhibited substantially reduced CSNA as compared to the population with untreated CHF, epidural RTX treatment showed substantially lower CSNA than epicardial RTX treatment.

### Example 6

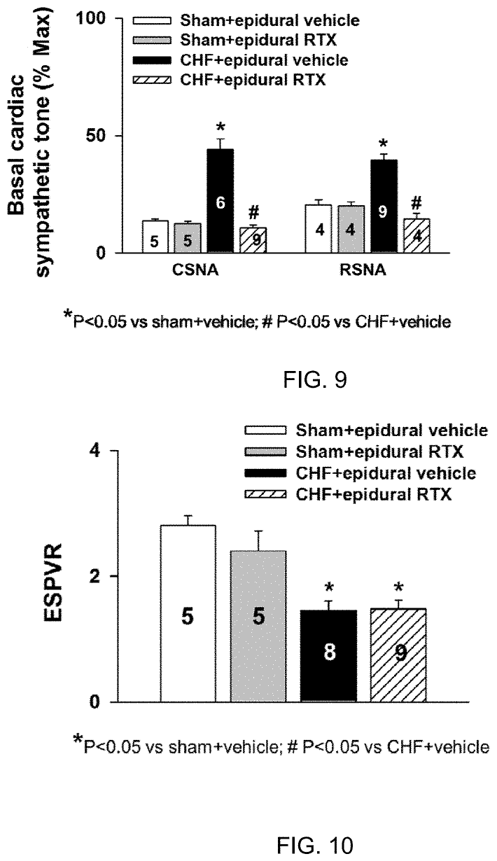

FIG. 9 shows experimental results for a rat study in accordance with an embodiment of the present application. FIG. 9 shows experimental data from a rat model showing basal cardiac sympathetic tone for cardiac sympathetic nerve activity (CSNA) and renal sympathetic nerve activity (RSNA) for sham and vehicle-only, sham and RTX, CHF and vehicle-only, and CHF with RTX populations. In each case, administration of vehicle or RTX plus vehicle was epidural. Statistically significant values against the sham with vehicle population are indicated by an asterisk (*), and statistically significant values against the CHF with vehicle-only population are indicated by a number sign (#). Both significance measures were at the P<0.05 level. FIG. 9 shows basal cardiac sympathetic tone for both cardiac sympathetic nerve activity (CSNA) and renal sympathetic nerve activity (RSNA). Induced CHF resulted in a substantial increase in cardiac sympathetic tone, which was not exhibited by the rat population treated with RTX. The difference in values for the CHF with RTX treatment population as compared to the untreated CHF population was statistically significant, while the difference between CHF with RTX treatment population and the non-CHF population was not statistically significant.

### Example 7

FIG. 10 shows experimental results for a rat study in accordance with an embodiment of the present application. FIG. 10 shows experimental data from a rat model showing end-systolic pressure volume relationship (ESPVR) for sham and vehicle-only, sham and RTX, CHF and vehicle-only, and CHF with RTX administration populations. In each case, administration of vehicle or RTX and vehicle was epidural. Statistically significant values (at the P<0.05 level) against the sham with vehicle population are indicated by an asterisk (*). FIG. 10 shows end-systolic pressure volume relationship (ESPVR), which correlates to systolic function of the heart. CHF corresponds to a reduction in ESPVR, with appeared unaffected by epidural RTX. A similar result has been found for epicardial RTX treatment (Wang et al. (2014) Hypertension 64(4):745-75).

### Example 8

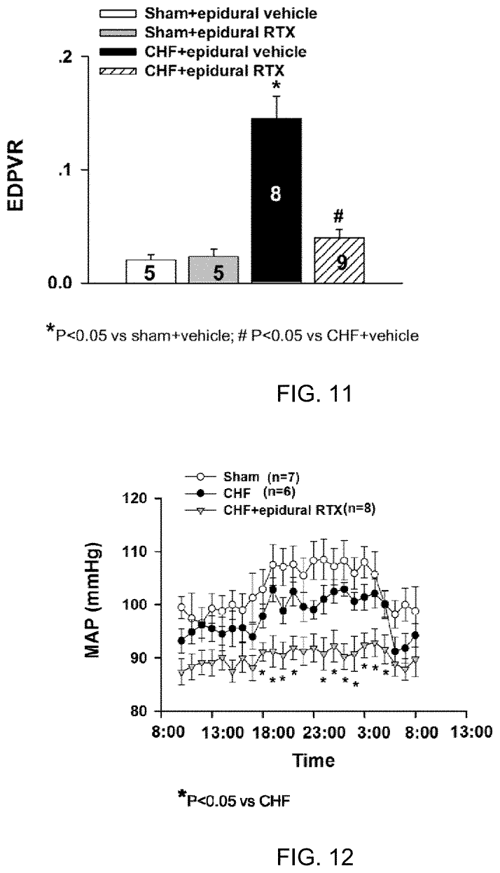

FIG. 11 shows experimental results for a rat study in accordance with an embodiment of the present application. FIG. 11 shows experimental data from a rat model showing the end diastolic pressure volume relationship (EDPVR) for sham and vehicle-only, sham and RTX, CHF and vehicle-only, and CHF with RTX populations. In each case, administration of vehicle or RTX and vehicle was epidural. Statistically significant values against the sham with vehicle population are indicated by an asterisk (*), and statistically significant values against the CHF with vehicle-only population are indicated by a number sign (#). Both significance measures were at the P<0.05 level.

FIG. 11 shows end diastolic pressure volume relationship (EDPVR), which correlated to diastolic function of the heart. CHF corresponds to an increase in EDPVR. FIG. 11 shows that the EDPVR reported for the population with CHF and RTX treatment was statistically significantly less than for the population with untreated CHF, while the EDPVR difference between the CHF population with RTX treatment and the population without CHF was not statistically significant.

### Example 9

FIG. 12 shows experimental results for a rat study in accordance with an embodiment of the present application. FIG. 12 shows experimental data from a rat model showing the mean arterial pressure (MAP) over 24 hours for sham and vehicle-only, CHF and vehicle-only, and CHF with epidural RTX administration populations. For each population, n=6-8. The study was conducted for a 10-12 week period after the myocardial infarction. FIG. 12 shows mean arterial pressure (MAP) over 24 hours. Treatment with epidural RTX corresponds to a lower MAP for the CHF rat population.

### Treatment of Hypertensive and Pre-Hypertensive Subjects.

Experimental data also showed successful treatment for subjects with hypertension and pre-hypertension (i.e., mild hypertension or early hypertension) using a rat model. The hypertensive model is a genetic spontaneously hypertensive rat (SHR). The early hypertension rats were treated at 8 weeks at age, reflecting a population in which blood pressure was only minimally elevated at the beginning of the study. Examples 10 and 11 illustrate treatment of subjects with hypertension and pre-hypertension (i.e., mild hypertension or early hypertension) and without induced CHF. For these subjects, epidural administration of RTX may be associated with an absolute reduction in blood pressure. Additionally, subjects treated with RTX may show less increase in blood pressure over time, as compared to a control population. Or, treatment may be associated with both an absolute reduction in pressure and lessened increase over time.

### Example 10

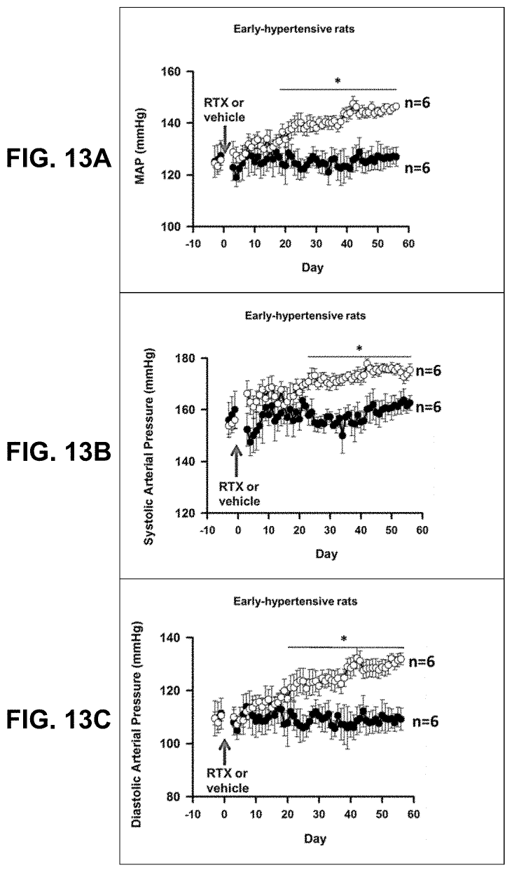

FIG. 13A-13C show experimental results in a study using an early-hypertensive rat model. FIG. 13A shows mean arterial pressure (MAP) from experimental data from a rat model featuring early hypertensive (i.e., prehypertensive or mildly hypertensive) subjects, both with RTX treatment and a control population with vehicle treatment only. Open circles indicate the control population, while filled circles indicate the RTX-treated population. Epidural administration of RTX was by injection on Day 0, as indicated by the arrow on each graph. The asterisk (*) and bar indicate data significantly different as between the two populations. FIG. 13B shows systolic arterial pressure from experimental data from a rat model featuring early hypertensive (i.e., prehypertensive or mildly hypertensive) subjects, both with RTX treatment and a control population with vehicle treatment only. Open circles indicate the control population, while filled circles indicate the RTX-treated population. Epidural administration of RTX was by injection on Day 0, as indicated by the arrow on each graph. The asterisk (*) and bar indicate data significantly different as between the two populations. FIG. 13C shows diastolic arterial pressure from experimental data from a rat model featuring early hypertensive (i.e., prehypertensive or mildly hypertensive) subjects, both with RTX treatment and a control population with vehicle treatment only. Open circles indicate the control population, while filled circles indicate the RTX-treated population. Epidural administration of RTX was by injection on Day 0, as indicated by the arrow on each graph. The asterisk (*) and bar indicate data significantly different as between the two populations.

FIG. 13A shows the mean arterial pressure (MAP) measured during the above study, which decreased slightly after RTX injection. More significantly, however, the rat population that received RTX injections exhibited an approximately steady MAP over the period of the study, while the MAP reported by the vehicle-only population continued to increase over that period. As a result, the MAP for the RTX-treated population was significantly lower at and after Day 20, as indicated by the asterisk and line in FIG. 13A.

FIG. 13B shows systolic arterial pressure measured during the above study. FIG. 13B shows that systolic arterial pressure decreased slightly within a few days after RTX injection, before returning to approximately the same pressure, and maintaining that pressure throughout the study period. By contrast, the systolic arterial pressure of the vehicle-only population continued to increase over the period of the study. By about Day 23 and thereafter, the RTX-treated population exhibited a significantly lower pressure than the control population.

FIG. 13C shows diastolic arterial pressure measured during the above study. FIG. 13C shows that diastolic arterial pressure remained approximately steady over the study period in the RTX-treated population. By contrast, the diastolic arterial pressure of the vehicle-only population continued to increase over the period of the study. By about Day 22 and thereafter, the RTX-treated population exhibited a significantly lower pressure than the control population.

### Example 11

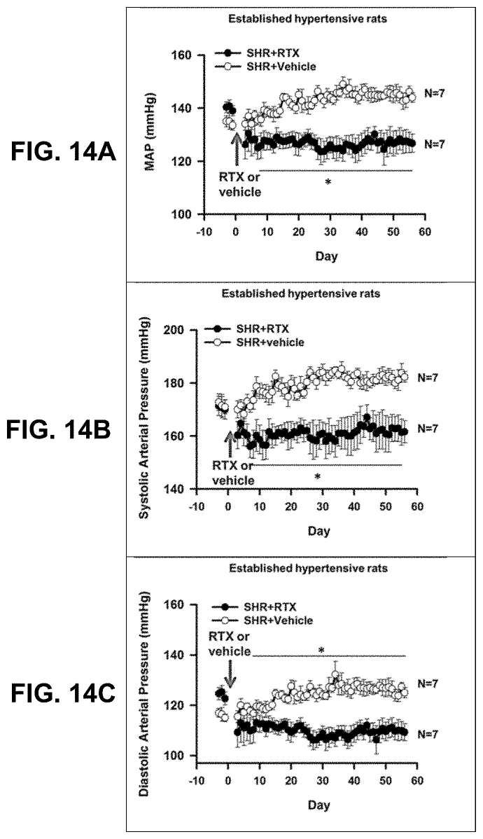

FIG. 14A-14C show further experimental results in a study using an (SHR) model. FIG. 14A shows mean arterial pressure (MAP) from experimental data from a spontaneously hypertensive rat (SHR) model with established hypertension, including a population treated with RTX and a control population treated with vehicle only. The established hypertensive rats were treated at 16 weeks at age, reflecting a population in which blood pressure was elevated at the beginning of the RTX administration. Open circles indicate the control population, while filled circles indicate the RTX-treated population. Epidural administration of RTX was by injection on Day 0, as indicated by the arrow on each graph. In each case, the asterisk (*) and bar indicate data significantly different as between the two populations. FIG. 14B shows systolic arterial pressure from experimental data from a spontaneously hypertensive rat (SHR) model, including a population treated with RTX and a control population treated with vehicle only. Open circles indicate the control population, while filled circles indicate the RTX-treated population. Epidural administration of RTX was by injection on Day 0, as indicated by the arrow on each graph. In each case, the asterisk (*) and bar indicate data significantly different as between the two populations. FIG. 14C shows diastolic arterial pressure from experimental data from a spontaneously hypertensive rat (SHR) model, including a population treated with RTX and a control population treated with vehicle only. Open circles indicate the control population, while filled circles indicate the RTX-treated population. Epidural administration of RTX was by injection on Day 0, as indicated by the arrow on each graph. In each case, the asterisk (*) and bar indicate data significantly different as between the two populations. These subjects were not treated to induce myocardial infarction. The data allowed for comparison of SHR rats provided injection of the vehicle only (n=7) to those treated by injection of both vehicle and RTX (n=7).

FIG. 14A shows that mean arterial pressure (MAP) showed some reduction in rats treated with RTX within days of the injection, as compared to the baseline level indicated by the first several days of testing before the injection. This reduced MAP was observed throughout the 55-day period of the study. By about Day 8, the reduction was significant as compared to the vehicle-only population, as indicated by the asterisk and line in FIG. 14A.

That significant difference was observed throughout the remainder of the study. Additionally, the vehicle-only population experienced an increasing trend in MAP, which was not observed in the RTX-treated population. FIG. 14B shows the systolic arterial pressure for the same population. Here again, the RTX-treated population reported a decrease after injection, as compared to the baseline level indicated by the first several days of testing before the injection. This reduced blood pressure was maintained throughout the remainder of the study. The RTX-treated population exhibited a significant difference over the vehicle-only population by about Day 7 and thereafter. Additionally, the vehicle-only population exhibited a greater increase in blood pressure over the course of the study. FIG. 14C shows the diastolic arterial pressure for the same population. Here again, the RTX-treated population reported a decrease after injection, as compared to the baseline level indicated by the first several days of testing before the injection. This reduced blood pressure was maintained throughout the remained of the study. The RTX-treated population exhibited a significant difference over the vehicle-only population by about Day 8 and thereafter. Additionally, the vehicle-only population exhibited a greater increase in blood pressure over the course of the study.

Example 11 showed that established hypertensive rats responded to RTX treatment. Before treatment, the average MAP of the established hypertensive rats was about 10-15 mmHg higher than the average MAP of the early hypertensive rats. Compare FIG. 14A with FIG. 13A. Following treatment, both the established and early hypertensive rats exhibited similar MAP, approximately 125-130 mmHg. These results demonstrate the advantages for both early and established hypertensive subjects.

### Example 12

Example 12 describes an experiment in which RTX administration was made to the lumbar region of a rat population (L2-L5). Example 12 shows that administration to the lumbar region does not achieve the sustained treatment of hypertension achieved by administration to the first through fourth thoracic vertebral levels.

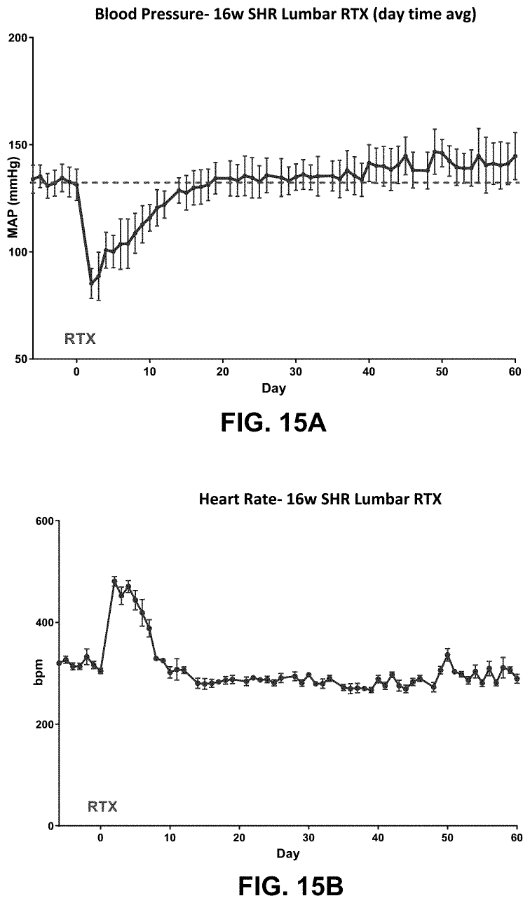

FIG. 15A shows mean arterial pressure (MAP) in mmHg, from experimental data from an established hypertensive rat model treated with RTX via lumbar administration in the L2-L5 region showing a period of seven days before administration and 60 days after administration, with RTX administration made on Day 0. FIG. 15A shows that MAP was reduced immediately following lumbar injection, but began to increase thereafter and returned to the original (pre-injection) baseline at about 15-20 days after treatment. Blood pressure continued to increase above the pre-injection baseline level by the end of the study period.

FIG. 15B shows heart rate (beats per minute) from experimental data from an established hypertensive rat model treated with RTX via lumbar administration in the L2-L5 region showing a period of seven days before administration and 60 days after administration, with RTX administration made on Day 0. FIG. 15B shows an increase in heart rate following RTX injection which subsides over about 2-10 days after injection.

Additionally, an immunofluorescence study for TRPV1 afferents was made following administration at the L2-L5 levels. With the L2-L5 levels, the DRG neurons showed elimination of most TRPV1 afferents following treatment. L1 exhibited robust fluorescence, showing that many TRPV1 afferents remained. Within the T1-T5 levels, no reduction in TRPV1 afferents was shown following the L2-L5 injection. Within the heart, TRPV1 fluorescence remained on the surfaces of the myocardium and within the cardiac tissue. The immunofluorescence study showed that the effects of the lumbar administration were localized, and did not lead to significant denervation in cardiac tissue or within non-treated vertebral levels.

Example 12 demonstrates that the sustained reduced hypertension associated with treatment to the T-T4 vertebral levels was not observed following administration to the L2-L5 vertebral levels. Further, the immunofluorescence study confirms that TRPV1 denervation was localized following L2-L5 treatment.

### Addressing Transient Blood Pressure and Heart Rate Elevation

The experiments performed in accordance with the present disclosure have demonstrated that administration of a TRPV1 agonist, such as RTX, as described herein, may cause a transient increase in blood pressure, heart rate, or both. This transient increase may occur following injection of the RTX and may last for several minutes, such as about five or ten minutes. This increase may have a deleterious effect on patients, and particularly on patients with underlying cardiac conditions such as hypertension, which may be expected to include many patients who may receive the present therapy.

The experiments performed in accordance with the present disclosure have also demonstrated that the transient elevated blood pressure and heart rate may be reduced or eliminated by means of pre-treatment with an opioid receptor agonist, more particularly with a μ-opioid receptor agonist. As described below, in Example 14, tests have been performed using the μ-opioid receptor agonist fentanyl. This administration was found to reduce or eliminate transient blood pressure and heart rate elevation.

While not wishing to be bound to any particular theory, the administration of a μ-opioid receptor agonist may result in activation of opioid receptors of the dorsal horn of the spinal column, thereby inhibiting transient sympatho-excitation. Additionally, the agonist may block pain input associated with the RTX administration.

### Example 13

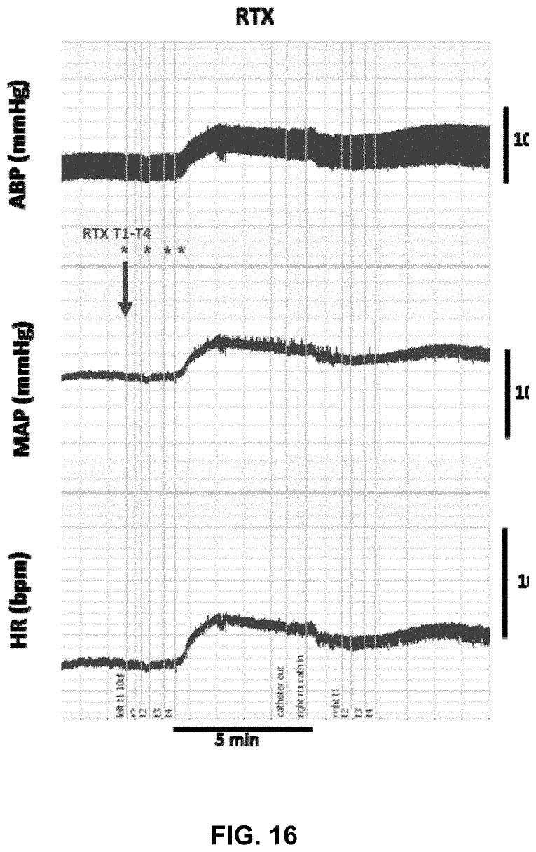

Example 13 demonstrates that pre-treatment of a subject with an opioid receptor agonist may be used to control short-term increases in average blood pressure, MAP, and heart rate observed following RTX injection. In the present context, epidural RTX administration to the T1-T4 vertebral levels may cause a short-term increase in blood pressure and heart rate. FIG. 16 shows experimental results following administration at the T1-T4 vertebral level, with the time of each injection indicated by the asterisks on the chart. The results demonstrated that ABP, MAP, and heart rate showed substantial short term increases, which extended over a period of several minutes following injection. This short-term increase may be associated with discomfort for the patient, and, in extreme cases, may be harmful to the patient, especially to a patient who already exhibits extreme high blood pressure or other cardiac conditions.

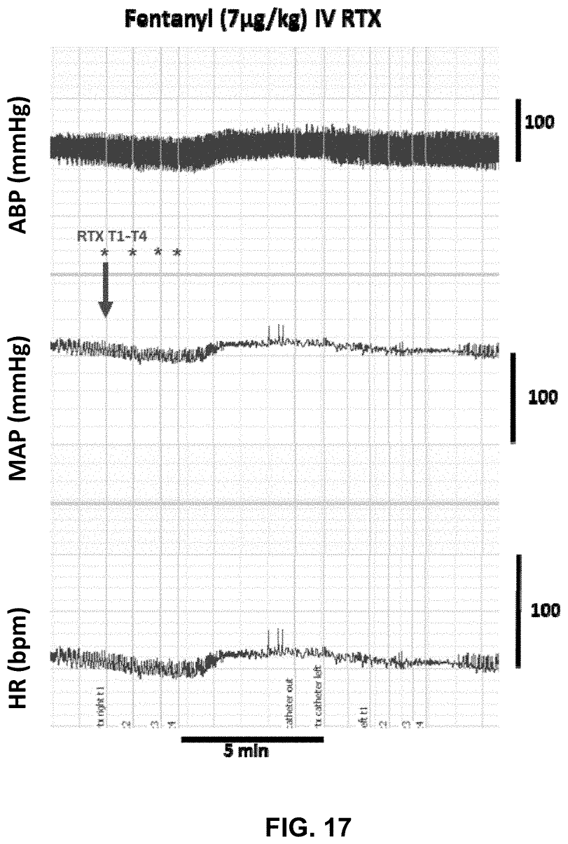

Example 13 shows that this short-term blood pressure and heart rate increase may be alleviated or eliminated by pre-treatment of the patient using fentanyl. In the present study, pre-treatment was performed using both intravenous and intraperitoneal injection. An appropriate form of administration may be selected based upon the subject to be treated. For example, in a human patient, intravenous administration may generally be preferred. FIG. 17 shows pre-treatment by 7 μg/kg intravenous (IV) fentanyl. Again, the asterisks indicate the times at which RTX injection was made. The results demonstrated that ABP, MAP, and heart rate showed little or no short-term increase following RTX administration in the pre-treated subject, especially in comparison to the increases observed in subjects that were not administered fentanyl (FIG. 16).

### Example 14

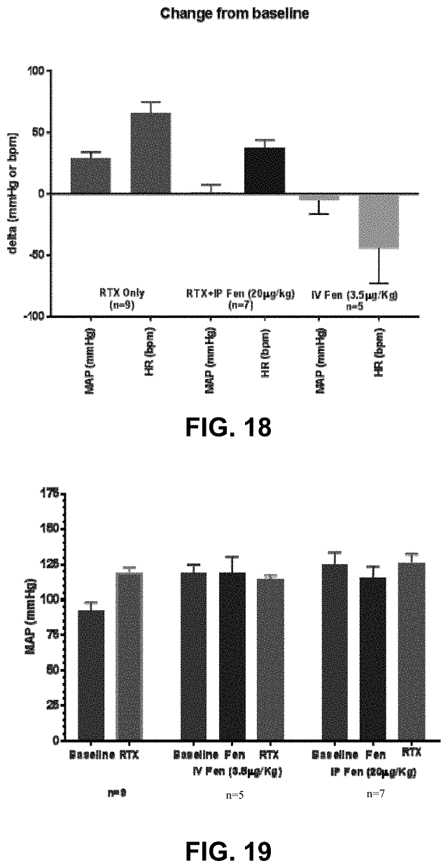

Example 14 further illustrates opioid pre-treatment before RTX administration as a means of controlling short-term blood pressure and heart rate elevation. The study used a hypertensive rat model. In the study, one group received no pre-treatment. A second group received a 20 μg/kg interperitoneal fentanyl pre-treatment. A third group received a 3.5 μg/kg intravenous fentanyl pre-treatment. FIG. 18 shows the changeover baseline for MAP and heart rate measurements for each of the three populations. The results showed that the population without pre-treatment exhibited an average short-term increase of about 30 mmHg and 70 bpm. The population that received 20 μg/kg intraperitoneal fentanyl pre-treatment demonstrated a negligible increase in MAP and significantly less increase in heart rate as compared to the population without pre-treatment. The population that received a 3.5 μg/kg intravenous fentanyl pre-treatment exhibited a reduction in MAP and heart rate.

FIG. 19 shows the absolute numbers for MAP for each population treated. FIG. 19 shows heart rate before treatment (baseline), after pre-treatment (“Fen,” in the case of the pre-treated populations), and after RTX administration. FIG. 19 shows that the administration of the opioid receptor agonist alone resulted in a small decrease in blood pressure. However, a decrease in MAP before RTX treatment was not necessary to achieve a moderated post-RTX reaction. That is, administration of the opioid receptor agonist may be established such that the patient's blood pressure remains relatively steady. Reduced fluctuation in blood pressure may provide further benefit to the patient in addition to the benefit associated with the absolute reduction.

The results shown in Example 14 demonstrate that pre-treatment by fentanyl may drastically reduce or even reverse the short-term blood pressure and heart rate increase associated with RTX injection, even to the point of achieving a short-term reduction in blood pressure and heart rate. The results demonstrate that both intravenous and intraperitoneal treatment may be effective, and that the dosage required varies significantly between the two forms of administration. The results indicated that intravenous administration requires less fentanyl than does intraperitoneal administration.

### Incorporation by Reference

The contents of all cited references (including literature references, patents, patent applications, and websites) that may be cited throughout this application are hereby expressly incorporated by reference in their entirety, as are the references cited therein. The practice of the present invention will employ, unless otherwise indicated, conventional techniques of surgery, cardiology, radiology, and interventional radiology, which are well known in the art.

### Equivalents

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

1. (canceled)

2. (canceled)

3. (canceled)

4. (canceled)

5. (canceled)

6. (canceled)

7. (canceled)

8. (canceled)

9. (canceled)

10. (canceled)

11. A method of decreasing blood pressure in a subject with high blood pressure, said method comprising:
administering resiniferatoxin to an epidural space located at one or more of the first through fourth thoracic vertebral levels of the subject.

12. The method of claim 11, wherein the blood pressure is systolic blood pressure.

13. The method of claim 11, wherein the blood pressure is diastolic blood pressure

14. A method of treating a cardiovascular condition in a subject, said method comprising administering resiniferatoxin to an epidural space at one or more of the first through fourth thoracic vertebral levels of the subject.

15. The method of claim 14 wherein the subject is a human.

16. The method of claim 14, wherein the cardiovascular condition is congestive heart failure.

17. The method of claim 14, wherein the cardiovascular condition is scarring of the subject's myocardium.

18. The method of claim 14, wherein the cardiovascular condition is selected from a group consisting of hypertension, prehypertension, or mild hypertension.

19. The method of claim 14, wherein the cardiovascular condition is selected from a group consisting of at least one of severe hypertension and refractory hypertension.

20. The method of claim 14, wherein the resiniferatoxin is administered to the epidural space proximate a first thoracic vertebra.

21. The method of claim 14, wherein the resiniferatoxin is administered to the epidural space proximate a second thoracic vertebra.

22. The method of claim 14, wherein the resiniferatoxin is administered to the epidural space proximate a third thoracic vertebra.

23. The method of claim 14, wherein the resiniferatoxin is administered to the epidural space proximate a fourth thoracic vertebra.

24. (canceled)

25. A method of preventative treatment of a subject, the subject having pre-hypertension or mild hypertension, said method comprising administering resiniferatoxin to an epidural space proximate a thoracic vertebra of the subject.

26. The method of claim 14, wherein the resiniferatoxin is administered at an amount of more than about 0.06 micrograms and less than about 30 micrograms.

27. The method of claim 14, wherein the administering comprises administering a solution comprising 0.6-10 micrograms of resiniferatoxin per milliliter of solution.

28. The method of claim 27, wherein the solution is administered at a volume of more than about 100 microliters and less than about 3 milliliters at each of said one or more vertebral levels.

29. A method of treating a cardiovascular condition in a subject, said method comprising:
administering an opioid receptor agonist to the subject; and
administering resiniferatoxin to an epidural space located at one or more of the first through fourth thoracic vertebral levels of the patient.

30. The method of claim 29 wherein the subject is a human.

31. The method of claim 29, wherein the opioid receptor is a μ-opioid receptor.

32. The method of claim 29, wherein the opioid receptor agonist is an opioid.

33. The method of claim 32, wherein the opioid is fentanyl.

34. The method of claim 33, wherein the fentanyl is administered in an amount corresponding to 50-100 μg fentanyl per kg of weight of the subject per 12 hours.

35. The method of claim 29 wherein the administration of the opioid receptor agonist is intravenous or intraperitoneal.

36. The method of claim 11, wherein the resiniferatoxin is administered at an amount of more than about 0.06 micrograms and less than about 30 micrograms.

37. The method of claim 11, wherein the administering comprises administering a solution comprising 0.6-10 micrograms of resiniferatoxin per milliliter of solution.

38. the method of claim 38, wherein the solution is administered at a volume of more than about 100 microliters and less than about 3 milliliters at each of said one or more vertebral levels.

39. The method of claim 14, wherein the cardiovascular condition is chronic heart failure.

40. The method of claim 14, wherein the epidural administration of resiniferatoxin provides improved left ventricle end diastolic pressure relative to epicardial administration.

41. The method of claim 14, wherein the epidural administration of resiniferatoxin provides at least comparable efficacy to epicardial administration with a reduction in potential adverse effects relative to epicardial administration.

42. The method of claim 14, wherein the epidural administration of resiniferatoxin provides cardiac sympathetic afferent ablation with a duration comprising about 6 months.

43. The method of claim 14, wherein the epidural administration of resiniferatoxin provides cardiac sympathetic afferent ablation with a duration greater than the duration provided by epicardial administration.
